(19) United States
(12) Patent Application Publication
(10) Pub. No.: US 2009/0281488 A1
(43) Pub. Date: Nov. 12, 2009

(54) PTCA GUIDING CATHETER WITH EXTRA LUMEN FOR CONTINUOUS MEDICATION INFUSION

(75) Inventor: David Rott, Modi'in (IL)

Correspondence Address: Fleit Gibbons Gutman Bongini & Bianco PL, 21355 EAST DIXIE HIGHWAY, SUITE 115, MIAMI, FL, 33180, US

(73) Assignee: HADASIT MEDICAL RESEARCH SERVICES AND DEVELOPMENT LTD., Jerusalem (IL)

(21) Appl. No.: 12/297,317

(22) PCT Filed: Apr. 16, 2007
(86) PCT No.: PCT/IL07/00480

§ 371 Date: Oct. 16, 2008

Related U.S. Application Data
(60) Provisional application No. 60/792,343, filed on Apr. 17, 2006.

(51) Int. Cl. A61M 29/00

(52) U.S. Cl. 604/96.01

(57) ABSTRACT

A multi-lumen guiding catheter for PTCA is disclosed. The guiding catheter comprises of a tubular body having a first and at least one second continuous lumen there-through from a proximal end to a distal end thereof The first lumen is having a cross-section and a length, the cross-section of the lumen is being large enough to permit passage there-through of a balloon of the balloon dilatation catheter The at least one second continuous lumen is positioned either in a concentric or eccentric manner in respect to the first lumen, and is adapted provide continuous intra-coronary infusion of a flowing matter A method of continuous intra-coronary infusion of flowing matter thorough a guiding catheter for PTCA is also presented. It comprises of steps of obtaining the aforesaid guiding catheter, and then, administrating a flowing matter thorough this second lumen from its proximal end.

### FIELD OF THE INVENTION

The present invention relates to Guiding Catheter which allows for constant intra-vascular infusion rate of medication fluids etc to achieve a steady state.

### BACKGROUND OF THE INVENTION

Percutaneous transluminal coronary angioplasty (PTCA) Guiding Catheter allows guidewire, PTCA balloon catheter and stent insertion to treat coronary stenosis and/or restenosis. It also allows administration of contrast dye as well as medications given as intra-coronary boluses. However it does not allow administration of medication at a constant rate to achieve steady state and constant effect during the procedure. If a procedure requires administration of medication at a constant rate, it must be infused systemically, even if the drug effect is needed intra-coronary only, and thus intra coronary infusion would suffice. Intra coronary infusion may allow lower drug dose with better local effect and fewer complications.

### SUMMARY OF THE INVENTION

It is one object of the invention wherein a multi-lumen guiding catheter for PTCA is provided. The guiding catheter comprises a tubular body having a first and at least one second continuous lumen there-through from a proximal end to a distal end thereof. The first lumen has a cross-section and a length, the cross-section of the lumen is large enough to permit passage there-through of a balloon of the balloon dilatation catheter. The at least one second continuous lumen there-through from a proximal end to a distal end thereof. The second lumen is positioned either in a concentric or eccentric manner in respect to the first lumen, and is adapted to provide, at least temporarily, continuous and constant-rate intra-coronary infusion of a flowing matter.

It is another object of the invention wherein the aforesaid at least one second continuous lumen is additionally comprised of at least one stopcock, which controls the flow of flowing matter in said lumen.

It is another object of the invention wherein the flowing matter is selected from one or more medicaments, contrast dyes or any combination thereof.

Another object of the invention is to disclose a method of continuous intra-coronary infusion of flowing matter thorough a guiding catheter for PTCA. The method comprises of steps of obtaining the guiding catheter as defined in any of the above, and then, administrating a flowing matter thorough this second lumen from its proximal end in a constant-rate such that a steady state is achieved. Optimally, the flowing matter is selected from one or more medicaments, contrast dyes or any combination thereof.

It is another object of the invention wherein the method additionally comprises the step or steps of regulating of the administration of said flowing matter, by obtaining at least one stopcock, which adapted to control the flow of flowing matter in said second lumen.

### BRIEF DESCRIPTION OF THE DRAWINGS

In order to understand the invention and to see how it may be implemented in practice, a plurality of embodiments will now be described, by way of non-limiting example only, with reference to the accompanying drawings, in which FIGS. 1 and 2 describe two possible configurations of the catheter:

FIG. 1 schematically describes in an out-of-scale manner the second (extra) lumen is in the circumference of the catheter around the first (conventional) lumen; and

FIG. 2 schematically describes in an out-of-scale manner the extra lumen inside the conventional lumen.

### DETAILED DESCRIPTION OF PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a PTCA Guiding Catheter with Extra Lumen.

According to one embodiment of the present invention, the PTCA Guiding Catheter is a long, preferably flexible tube, especially made of soft plastic tube. It is in the scope of the invention wherein a new set of PTCA Guiding Catheters of conventional diameters, e.g., 6F, 7F, and 8F, and conventional configuration, e.g., Judkins, extra-backup, Amplatz etc is disclosed. The new PTCA set is equipped with an additional inner lumen that runs along the full length of the main lumen. This lumen allows continuous intra-coronary infusion of medications, e.g., Nitroglycerin, Thrombolytic agents, IIb/IIa inhibitors, anti-arrhythmic medications etc during the procedure without interrupting the procedure carried out using the main lumen. In both figures, the letter A represents the cross sectional view and the letter B represents the longitudinal view.

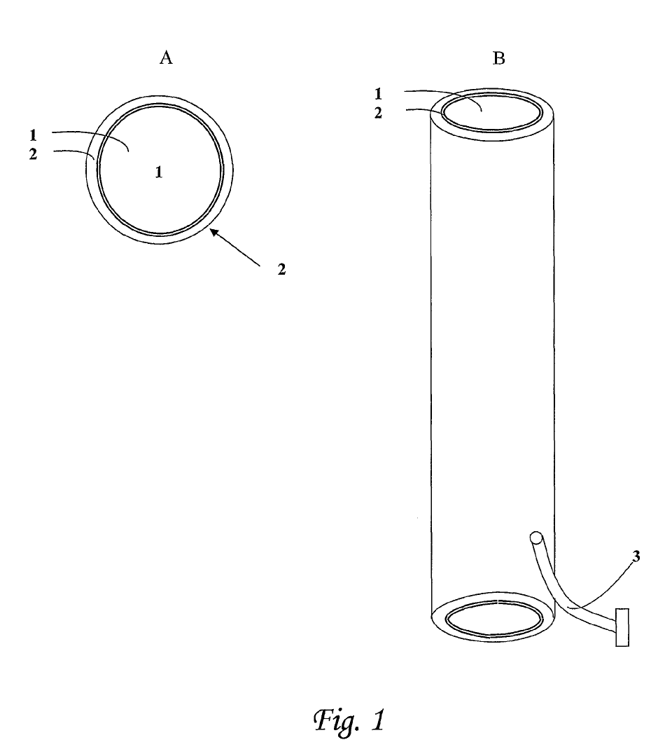

FIG. 1, schematically shows the conventional elongated lumen (1), wherein at least one extra lumen (2) is mounted circumferentially, e.g., either concentric or eccentric to the conventional lumen. The second lumen is adapted to allow continuous intra-coronary infusion of medications. A stopcock (3) preferably controls the flow of fluid in the extra lumen (2).

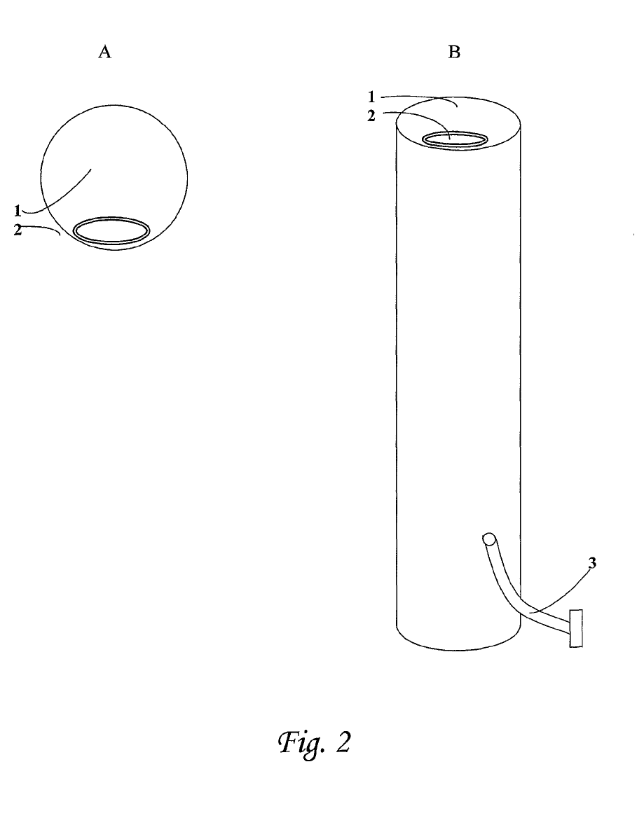

FIG. 2, schematically shows another embodiment of the present invention, wherein the extra lumen (2) is mounted inside the main lumen (1), preferably controlled by a stopcock (3).

1. A guiding catheter for PTCA, comprising:
a tubular body having a first and at least one second continuous lumen there-through from a proximal end to a distal end thereof; said first lumen having a cross-section and a length, the cross-section of said lumen being large enough to permit passage there-through of a balloon of the balloon dilatation catheter; said at least one second continuous lumen there-through from a proximal end to a distal end thereof; wherein said second lumen is positioned either in a concentric or eccentric manner in respect to said first lumen, and is adapted provide, at least temporarily, continuous and constant-rate intra-coronary infusion of flowing matter.

2. The guiding catheter for PTCA according to claim 1, wherein said at least one second continuous lumen additionally comprises at least one stopcock, which controls the flow of flowing matter in said lumen.

3. The guiding catheter for PTCA according to claim 1, wherein said flowing matter is selected from one or more medicaments, contrast dyes or any combination thereof.

4. A method of continuous and constant intra-coronary infusion of flowing matter thorough a guiding catheter for PTCA, comprising:
a. obtaining a tubular body having a first and at least one second continuous lumen there-through from a proximal end to a distal end thereof; said first lumen having a cross-section and a length, the cross-section of said lumen being large enough to permit passage there-through of a balloon of the balloon dilatation catheter; said at least one second continuous lumen there-through from a proximal end to a distal end thereof; wherein said second lumen is positioned either in a concentric or eccentric manner in respect to said first lumen, and is adapted provide, at least temporarily, continuous and constant-rate intra-coronary infusion of flowing matter; and,
b. administering said flowing matter thorough said at least one second lumen from its proximal end such that a steady state is achieved.

5. The method according to claim 1, additionally comprising regulating the administration of said flowing matter, by obtaining at least one stopcock, which adapted to control the flow of flowing matter in said second lumen.

6. The method according to claim 1, wherein said flowing matter is selected from one or more medicaments, contrast dyes or any combination thereof.
